(19) United States
(12) Patent Application Publication
(10) Pub. No.: US 2015/0282803 A1
(43) Pub. Date: Oct. 8, 2015

(54) SURGICAL DEVICE FOR REPAIRING TISSUE

(71) Applicant: Vishal M. Mehta, Naperville, IL (US)

(72) Inventor: Vishal M. Mehta, Naperville, IL (US)

(21) Appl. No.: 14/674,777

(22) Filed: Mar. 31, 2015

Related U.S. Application Data
(60) Provisional application No. 61/973,905, filed on Apr. 2, 2014.

(51) Int. Cl. A61B 17/04

(52) U.S. Cl. CPC A61B 17/0401; A61B 2017/0441; A61B 2017/0446; A61B 2017/0458

(57) ABSTRACT

A surgical device for repairing a damaged portion of labral tissue is provided. The surgical device includes a first anchor, a second anchor, and a continuous suture. The first anchor is adapted to be implanted in a first location of a bone fragment of a glenoid. The second anchor is adapted to be implanted in a second location of the bone fragment. The continuous suture is adapted to extend from the first anchor to the second anchor. The continuous suture is adapted to wrap around labral tissue between the first anchor and the second anchor to secure the labral tissue between the first anchor and the second anchor.

### CROSS REFERENCE TO RELATED APPLICATIONS

This application claims the benefit of Provisional Patent Application No. 61/973,905 entitled “Device for labral repair”, filed on Apr. 2, 2014, in the United States Patent and Trademark Office, incorporated herein by reference in its entirety.

### BACKGROUND

1. Technical Field

The embodiments herein generally relate to surgical devices, and, more particularly, a surgical device used for repairing tissue, such as labral tissue.

2. Description of the Related Art

Soft tissue such as ligaments and cartilage are generally attached to bone by small collagenous fibers which are strong, but tend to degenerate over time or tear due to injury or disease. A scapula or a shoulder blade, is a bone that connects a humerus (e.g., a bone in an upper arm) with a clavicle (e.g., a collar bone). A portion of the scapula, the glenoid, forms a socket which is congruent with the head of the humerus. A ring of fibrocartilage that runs around the glenoid into which the humeral head fits is called the labrum. The labrum deepens the glenoid cavity and effectively increases the surface of the shoulder joint while providing increased stability.

The glenoid labrum is a fibrocartilaginous rim attached around the margin of the glenoid cavity in the shoulder blade. The shoulder joint is considered a ball and socket joint. However, the glenoid fossa of the scapula (e.g., the socket) is shallow and small, covering at most only a third of the head of the humerus (e.g., the ball). The socket is deepened by the glenoid labrum. The base of the labrum is fixed to the circumference of the cavity, while the rounded free edge extends outwards from the glenoid.

Tearing of the labrum can occur from either acute trauma or repetitive shoulder motion such as in the sports of swimming and baseball. Acute trauma may be from dislocation of the shoulder, falling on an outstretched arm, direct blows to the shoulder etc. When the shoulder dislocates anteriorly, the humeral head is forced violently out of the socket. The joint capsule and ligaments of the front of the shoulder are put under significant tension, and either have to stretch out, or more commonly pull so hard on the labrum where they insert that the labrum detaches from the anterior glenoid bone surface. Surgery is focused on repairing the labrum back onto the glenoid rim, thereby restabilizing the joint.

### SUMMARY

This summary is provided to introduce a selection of concepts in a simplified form that are further described below in the detailed description. This summary is not intended to identify key or essential features of the claimed subject matter, nor is it intended to be used as an aid in determining the scope of the claimed subject matter. In view of the foregoing, an embodiment herein provides a surgical device for repairing labral tissue. The surgical device includes a first anchor, a second anchor, and a continuous suture. The first anchor is adapted to be implanted in a first location of a bone fragment of a glenoid. The second anchor is adapted to be implanted in a second location of the bone fragment. The continuous suture is adapted to extend from the first anchor to the second anchor. The continuous suture is adapted to wrap around labral tissue between the first anchor and the second anchor to secure the labral tissue between the first anchor and the second anchor.

In another embodiment, the surgical device further includes a third anchor that is adapted to be implanted in a third location of the bone fragment. The continuous suture is adapted to extend from the second anchor to wrap around labral tissue between the second anchor and the third anchor. In yet another embodiment, the continuous suture is adapted to secure the labral tissue between the second anchor and the third anchor. In yet another embodiment, the surgical device further includes a fourth anchor that is adapted to be implanted in a fourth location of the bone fragment. The continuous suture is adapted to extend from the third anchor to wrap around labral tissue between the third anchor and the fourth anchor. In yet another embodiment, the continuous suture is adapted to secure the labral tissue between the third anchor and the fourth anchor. In yet another embodiment, the continuous suture partially wraps around the labral tissue. In yet another embodiment, the continuous suture is adapted to extend from a first side of the first anchor to the second anchor. In yet another embodiment, the continuous suture is adapted to extend from a second side of the second anchor to a first side of the third anchor. In yet another embodiment, the continuous suture is a separate suture that connects the second anchor to the third anchor.

In another aspect, a surgical apparatus for repairing tears in soft tissue is provided. The surgical apparatus includes a first anchoring member, a second anchoring member, a continuous strand, and a third anchoring member. The first anchoring member is adapted to be implanted in a first location of a bone fragment. The second anchoring member is adapted to be implanted in a second location of the bone fragment. The continuous strand is adapted to extend from the first anchoring member to the second anchoring member. The continuous strand is adapted to wrap around tears in tissue between the first anchoring member and the second anchoring member to secure the tears in the soft tissue between the first anchoring member and the second anchoring member. The third anchoring member is adapted to be implanted in a third location of the bone fragment. The continuous strand extends from the second anchoring member to wrap around tears in the soft tissue between the second anchoring member and the third anchoring member to secure the tears in the soft tissue between the second anchoring member and the third anchoring member.

In yet another embodiment, the surgical apparatus further includes a fourth anchoring member that is adapted to be implanted in a fourth location of the bone fragment. The continuous strand extends from the third anchoring member to wrap around tears in the soft tissue between the third anchoring member and the fourth anchoring member. In another embodiment, the continuous strand is adapted to be attached to the fourth anchoring member to secure the tears of the soft tissue between the third anchoring member and the fourth anchoring member. In yet another embodiment, the continuous strand is a suture strand. In yet another embodiment, the tear in the soft tissue is a tear in labral tissue.

In yet another aspect, a method of repairing labral tissue is provided. The method includes the following steps: (i) implanting a first anchor, and a second anchor into a first location, and a second location, respectively, of a bone fragment along a glenoid, (ii) passing a first suture around the labral tissue at the first location of the bone fragment, (iii) attaching a first end of the first suture to the first location of the bone fragment using the first anchor, (iv) cutting the first end of the first suture from the first anchor to leave a second end of the first suture to wrap around the labral tissue, (v) wrapping the second end of the first suture around the labral tissue between the first anchor and the second anchor, (vi) passing a second suture around the labral tissue at the second location of the bone fragment, (vii) attaching (a) a first end and a second end of the second suture, and (b) the second end of the first suture to the second location of the bone fragment using the second anchor, and (viii) cutting (a) the second end of the first suture that is extending from the first anchor, and (b) the first end of the second suture from the second anchor to leave the second end of the second suture to wrap around the labral tissue.

In yet another embodiment, the method further includes the following steps: (i) implanting a third anchor at a third location of the bone fragment along the glenoid, (ii) wrapping the second end of the second suture around the labral tissue between the second anchor and the third anchor, (iii) passing a third suture around the labral tissue at the third location of the bone fragment, (iv) attaching (a) a first end and a second end of the third suture, and (b) the second end of the second suture to the third location of the bone fragment using the third anchor, and (v) cutting (a) the second end of the second suture that is extending from the second anchor, and (b) the first end of the third suture from the third anchor to leave the second end of the third suture to wrap around the labral tissue.

In another embodiment, the method further includes the following steps: (i) implanting a fourth anchor at a fourth location of the bone fragment along the glenoid, and (ii) wrapping the second end of the third suture around the labral tissue between the third anchor and the fourth anchor.

### BRIEF DESCRIPTION OF THE DRAWINGS

The embodiments herein will be better understood from the following detailed description with reference to the drawings, in which:

FIGS. 1A-1C illustrate sectional views of repairing soft tissue using a surgical device according to an embodiment herein;

FIG. 2 illustrates a perspective view of the continuous suture of FIG. 1 that extends from the first anchor according to an embodiment herein; and

FIGS. 3A and 3B are flow diagrams illustrating a method of repairing labral tissue using the surgical device of FIGS. 1A-1C according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

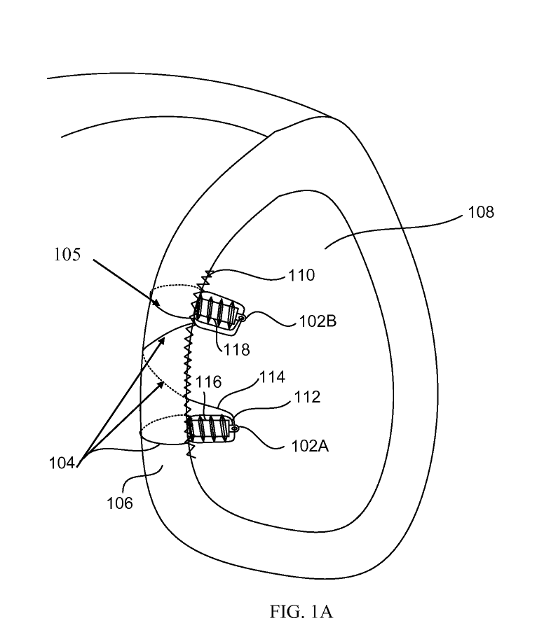

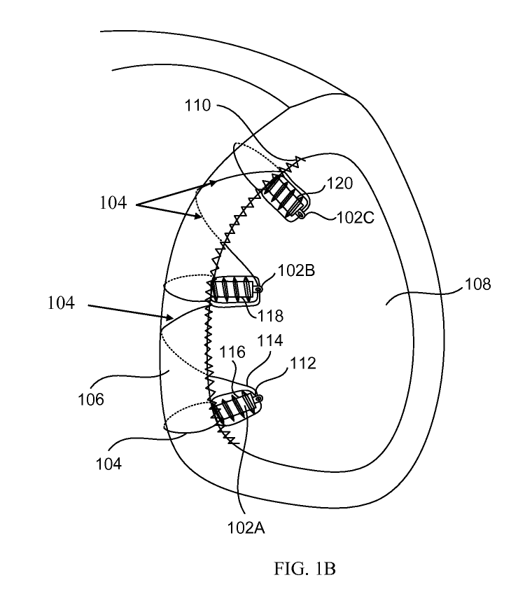

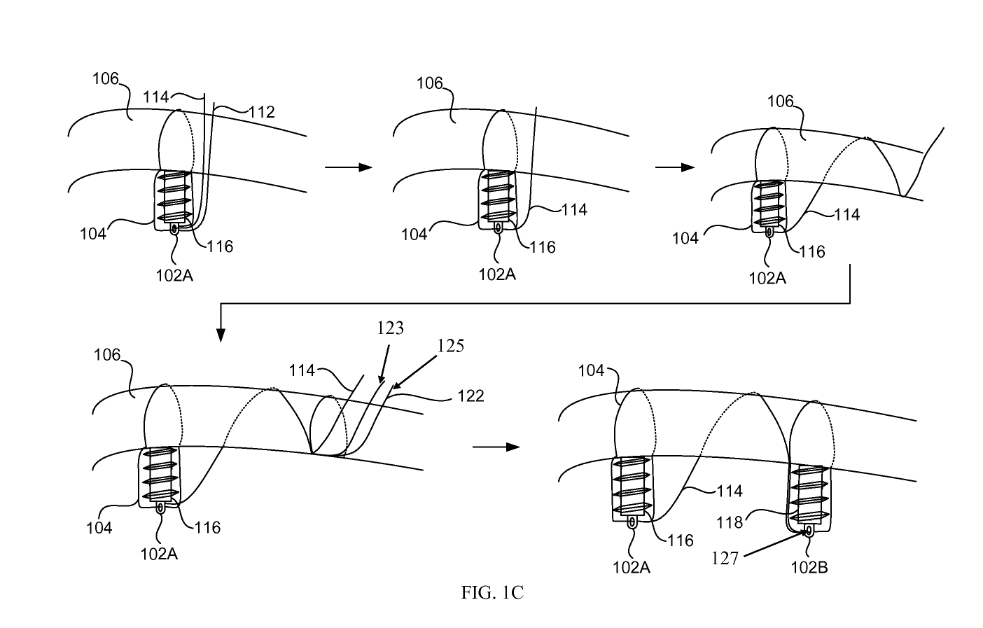

Pursuant to an exemplary scenario, there remains a need for a device and a surgical method that provides better support and strength to labral tissue. The embodiments herein achieve this by providing a device and a method for repairing a tear in soft tissue using two or more anchors and a continuous suture (e.g., a tape) that is attached to at least two anchors. Referring now to the drawings, and more particularly to FIGS. 1A through 3B, where similar reference characters denote corresponding features consistently throughout the figures, preferred embodiments are shown.

FIGS. 1A-1C illustrate sectional views of repairing soft tissue 106 using a surgical device according to an embodiment herein. The surgical device includes two or more anchors (102A, 102B-102C), and a continuous suture 104 (e.g., a tape) that is attached to at least two anchors (e.g., 102A, 102B). The two or more anchors (i.e. two or more anchoring members) are adapted to be implanted in a bone fragment 108. In one embodiment, the bone fragment 108 is a bone fragment of a glenoid. In another embodiment, the bone fragment 108 includes any part of the bone, and/or any parts of the anatomy. The two or more anchors (102A, 102B-102C) include a first anchor 102A, and a second anchor 102B. The first anchor 102A is adapted to be implanted in a first location (e.g., a fixation point) of the bone fragment 108. The second anchor 102B is adapted to be implanted in a second location of the bone fragment 108. In one embodiment, the soft tissue 106 is a labral tissue. In another embodiment, the soft tissue 106 includes any other type of tissue that needs to be secured.

The continuous suture 104 (e.g., a continuous strand or tape) is adapted to secure a damaged portion 110 of the labral tissue 106 attaches to the first anchor 102A. In one embodiment, the continuous suture 104 is a non-absorbable suture, and/or an absorbable suture. The non-absorbable suture/absorbable suture include a high-strength suture, and/or a polyester based suture. In one embodiment, the continuous suture 104 includes a first end 112 and a second end 114. The first end 112 of the continuous suture 104 may attach to a first side 116 of the first anchor 102A by being attached directly to the anchor 102A or being wedged between the anchor 102A and bone fragment 108. The continuous suture 104 is attached to the at least two anchors (i.e. the first anchor 102A, and the second anchor 102B) based on known attachment mechanisms such as interference fixation, or a locking mechanism within the anchors (e.g., the first anchor 102A, and the second anchor 102B). It is also appreciated that a separate suture 105 can loop around the labral tissue 106 that is separate and apart from the continuous suture 106.

The second end 114 of the continuous suture 104 is adapted to extend from the first anchor 102A to the second anchor 102B. In one embodiment, the second end 114 of the continuous suture 104 is adapted to extend from the first side 116 of the first anchor 102A. The second end 114 of the continuous suture 104 is adapted to wrap around the damaged portion 110 of the labral tissue 106 between the first anchor 102A and the second anchor 102B to secure the damaged portion 110 of the labral tissue 106 between the first anchor 102A and the second anchor 102B and the bone 108. In one embodiment, the second end 114 of the continuous suture 104 attaches to a second side 118 of the second anchor 102B to secure the damaged portion 110 of the labral tissue 106 between the first anchor 102A and the second anchor 102B.

In another embodiment, the second end 114 of the continuous suture 104 is pre-connected to the second anchor 102B, and the first end 112 of the continuous suture 104 is then connected to the first anchor 102A. In an alternative embodiment, both anchors (102A, B) are attached to the continuous suture 104, such that one of the anchors is looped around the labral tissue in order to secure the continuous suture 104 to the labral tissue 106.

In another embodiment as shown in FIG. 1B, the two or more anchors include a third anchor 102C that is adapted to be implanted in a third location of the bone fragment 108. The second end 114 of the continuous suture 104 is adapted to extend from the second anchor 102B to the third anchor 102C. In one embodiment, the continuous suture 104 is a separate suture that connects the second anchor 102B to the third anchor 102C, or the continuous suture 104 is a continuous piece that extends from the first anchor 102A. The second end 114 of the continuous suture 104 is adapted to wrap around the damaged portion 110 of the labral tissue 106 between the second anchor 102B and the third anchor 102C to secure the labral tissue 106 between the second anchor 102B and the third anchor 102C. In one embodiment, the continuous suture 104 is adapted to extend from the second side 118 of the second anchor 102B. In another embodiment, the second end 114 of the continuous suture 104 is attached to the third anchor 102C to secure the damaged portion 110 of the labral tissue 106 between the second anchor 102B and the third anchor 102C. In yet another embodiment, the second end 114 of the continuous suture 104 attaches to a first side 120 of the third anchor 102C. Likewise, the continuous suture 104 can also attach to the first sides of each anchor as well.

In yet another embodiment, a second suture 122 is passed around the labral tissue 106 at the second location of the bone fragment 108 which coincides with the second anchor 102B as shown in FIG. 1C. A first end 123 and a second end 125 of the second suture 122, and the second end 114 of the continuous suture 104 are attached to the second location of the bone fragment 108 using the second anchor 102B (not shown). The second end 114 of the continuous suture 104 that is extending from the first anchor 102A and the first end 123 of the second suture 122 are cut from the second anchor 102B to leave the second end 125 of the second suture 122 to wrap around the labral tissue 106. In yet another embodiment, the second anchor 102B includes an opening 127 though which the first end 123 and the second end 125 of the second suture 122, and the second end of the continuous suture 104 are passed through and secured at the second location of the bone fragment 108.

In yet another embodiment, the two or more anchors (102A, 102B-102C) include a fourth anchor (not shown in Figure) that is adapted to be implanted in a fourth location of the bone fragment 108. The second end 114 of the continuous suture 104 is adapted to extend from the third anchor 102C to the fourth anchor. In another embodiment, the second end 114 of the continuous suture 104 extends from the first side 120 of the third anchor 102C. The second end 114 of the continuous suture 104 is adapted to wrap around the damaged portion 110 of the labral tissue 106 between the third anchor 102C and the fourth anchor (e.g., a fourth anchoring member) to secure the damaged portion 110 of the labral tissue 106 between the third anchor 102C and the fourth anchor. In one embodiment, the second end 114 of the continuous suture 104 attaches to the fourth anchor to secure the damaged portion 110 of the labral tissue 106 between the third anchor 102C and the fourth anchor. In another embodiment, the second end 114 of the continuous suture 104 attaches to a second side of the fourth anchor.

In yet another embodiment, a third suture is passed around the labral tissue 106 at the third location of the bone fragment 108. A first end and a second end of the third suture (not shown in Figure), and the second end of the second suture 122 are attached to the third location of the bone fragment 108 using the third anchor 102C. The second end of the second suture 122 that is extending from the second anchor 102B, and the first end of the third suture are cut from the third anchor 102C to leave the second end of the third suture to wrap around the labral tissue 106. In yet another embodiment, the third anchor 102C includes an opening though which the first end and the second end of the third suture, and the second end of the second suture 122 are passed through and secured at the third location of the bone fragment 108.

The continuous suture 104 may wrap around the damaged portion 110 of the labral tissue 106 in a winding manner. The continuous suture 104 wraps around the damaged portion 110 of the labral tissue 106, and provides a constant and substantially uniform securing force along the length of the tear or the damaged portion 110 of the labral tissue 106. The continuous suture 104 secures the labral tissue 106 not only at sites of the two or more anchors (e.g., the first anchor 102A, the second anchor 102B, and the third anchor 102C), but also between the sites of the two or more anchors. In one embodiment, the continuous suture 104 does not wrap entirely (i.e. partially) around the damaged portion 110 of the labral tissue 106, such that the continuous suture 104 runs between the labral tissue 106 and the bone fragment 108 (e.g., the bone fragment of the glenoid). The two or more anchors may include an opening through which the suture (e.g., the continuous suture 104, the second suture 122, and the third suture) passes through the two or more anchors. In one embodiment, the surgical device includes an assembly that is adapted to insert the two or more anchors within the soft tissue 106 (i.e. the labral tissue) to be repaired.

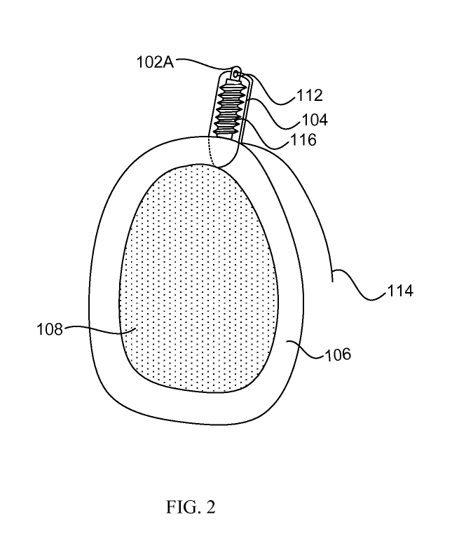

With reference to FIGS. 1A-1C, FIG. 2 illustrates a perspective view of the continuous suture 104 that extends from the first anchor 102A according to an embodiment herein. The first end 112 of the continuous suture 104 attaches to the first side 116 of the first anchor 102A and wraps around the labral tissue 106 in a winding manner. The second end 114 of the continuous suture 104 is extended from the first side 116 of the first anchor 102A to wrap around the damaged portion 110 of the labral tissue 106 between the first anchor 102A and the second anchor 102B. The second end 114 of the continuous suture 104 is then attached to a second side of the subsequent anchor (e.g. a second anchor 102B) to secure the damaged portion 110 of the labral tissue 106 between the first anchor 102A and the subsequent anchor. It is appreciated that the continuous suture 104 may run between every other or every third anchor (and the like) such that it does not directly attached two adjacent anchors together.

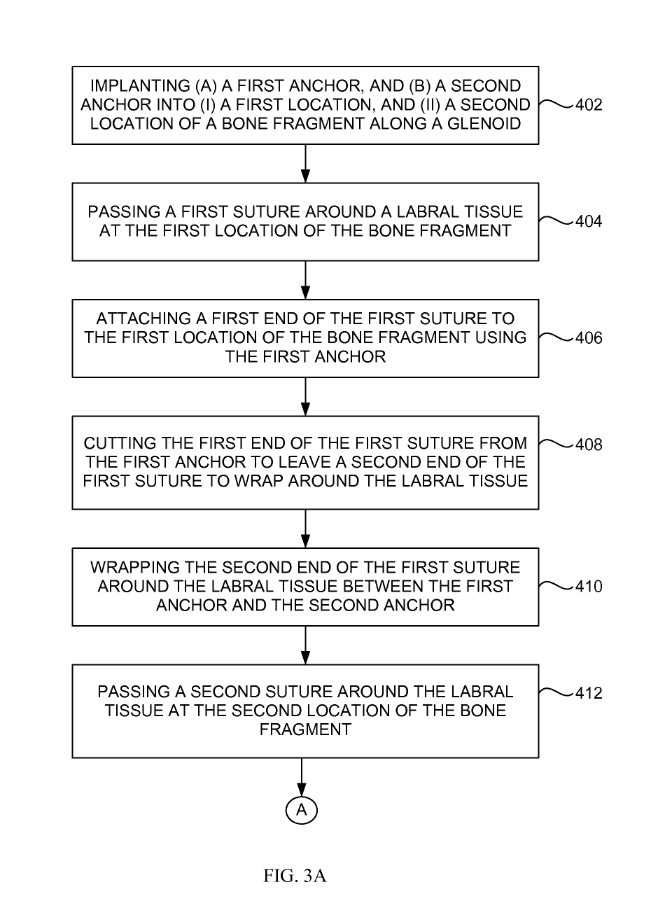

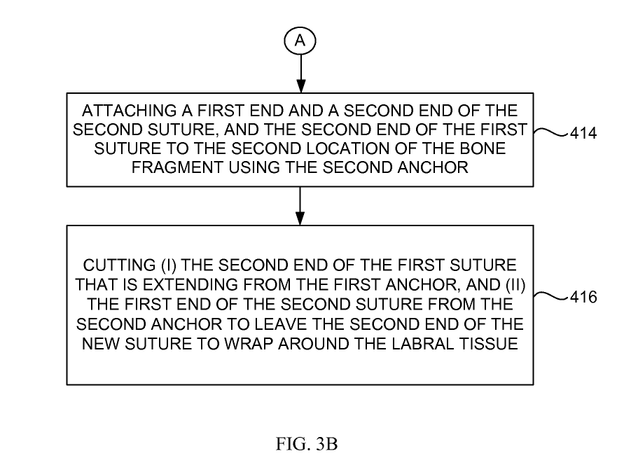

FIGS. 3A and 3B are flow diagrams illustrating a method of repairing the labral tissue 106 using the surgical device of FIGS. 1A-C and 2 according to an embodiment herein. At step 402, a first anchor 102A, and a second anchor 102B are implanted into (i) a first location, and (ii) a second location, respectively, of a bone fragment 108 along a glenoid. At step 404, a first suture (e.g., a continuous suture 104) is passed around the labral tissue 106 at the first location of the bone fragment 108. In one embodiment, the first suture 104 includes a first end 112 and a second end 114. At step 406, the first end 112 of the first suture 104 is attached to the first location of the bone fragment 108 using the first anchor 102A. The first end 112 of the first suture 104 is attached to a first side 116 of the first anchor 102A. At step 408, the first end 112 of the first suture 104 is cut from the first anchor 102A to leave the second end 114 of the first suture 104 to wrap around the labral tissue 106. At step 410, the second end 114 of the first suture 104 is wrapped around the labral tissue 106 between the first anchor 102A and the second anchor 102B. In one embodiment, the second end 114 of the first suture 104 is attached to the second anchor 102B to secure the labral tissue 106 between the first anchor 102A and the second anchor 102B. In another embodiment, the second end 114 of the first suture 104 is attached to a second side 118 of the second anchor 102B to secure the labral tissue 106 between the first anchor 102A and the second anchor 102B. At step 412, a second suture 122 is passed around the labral tissue 106 at the second location of the bone fragment 108. At step 414, (a) a first end 123 and a second end 125 of the second suture 122, and (b) the second end 114 of the first suture 104 are attached to the second location of the bone fragment 108 using the second anchor 102B. At step 416, the second end 114 of the first suture 104 that is extending from the first anchor 102A, and the first end 123 of the second suture 122 are cut from the second anchor 102B to leave the second end 125 of the second suture 122 to wrap around the labral tissue 106.

In one embodiment, the second end 125 of the second suture 122 that is attached to the second anchor 102B is extended form the second anchor 102B to wrap around the labral tissue 106. In another embodiment, instead of the second suture 122, the first suture (i.e., the continuous suture 104) is passed around the labral tissue 106 at the second location of the bone fragment 108.

In yet another embodiment, the method further includes the following steps: (i) implanting a third anchor 102C at a third location of the bone fragment 108 along the glenoid, (ii) wrapping the second end 125 of the second suture 122 around the labral tissue 106 between the second anchor 102B and the third anchor 102C, (iii) passing a third suture around the labral tissue at the third location of the bone fragment 108, (iv) attaching (a) a first end and a second end of the third suture, and (b) the second end of the second suture 122 to the third location of the bone fragment 108 using the third anchor 102C, and (v) cutting (a) the second end of the second suture 122 that is extending from the second anchor 102B, and (b) the first end of the third suture from the third anchor 102C to leave the second end of the third suture to wrap around the labral tissue 106. In one embodiment, the second end 125 of the second suture 122 is attached to a first side 120 of the third anchor 102C to secure the labral tissue 106 between the second anchor 102B and the third anchor 102C. In another embodiment, instead of the third suture, the first suture (i.e. the continuous suture 104) is passed around the labral tissue 106 at the third location of the bone fragment 108.

In yet another embodiment, the method further includes the following steps: (i) implanting a fourth anchor at a fourth location of the bone fragment 108 along the glenoid, and (ii) wrapping the second end of the third suture around the labral tissue 106 between the third anchor 102C and the fourth anchor. In yet another embodiment, the sutures (e.g., the first suture 104, the second suture 122, and the third suture) are attached to the at least two anchors (e.g., the first anchor 102A, the second anchor 102B, the third anchor 102C) based on known attachment mechanisms such as interference fixation, or a locking mechanism within the anchors (e.g., the first anchor 102A, the second anchor 102B, and the third anchor 102C).

It is also contemplated that the first suture 104 is first passed around a portion of the tissue (e.g., labrum) before the first anchor 102A is placed. Then the first suture 104 is fed through the opening 127 at the distal end of the first anchor 102A. Then the first suture 104 is wound around a portion of the tissue traveling away from the first anchor 102A. The first suture 104 is then fed through the opening 127 at the distal end of the second anchor 102B. A second suture 122 is fed around the tissue adjacent to the second anchor 102B, and passed through the opening 127 at the distal end of the second anchor 102B. Thus, the first suture 104 and the second suture 122 are both disposed through the opening 127 of the second anchor 102B. One end of the second suture 122 is then wound around a portion of the tissue traveling away from the second anchor 102B and the process is repeated with a third anchor 102C.

The continuous suture 104 wraps around the damaged portion 110 of the labral tissue 106 between the two or more anchors, and provides a constant and uniform securing force along the length of the tear or the damaged portion 110 of the labral tissue 106. The continuous suture 104 secures the labral tissue 106 not only at sites of the two or more anchors, but also between the sites of the two or more anchors.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. The methods of attaching two or more anchors with a continuous cuture can be varied as contemplated within this disclosure and it is appreciated that one or more attachment methods disclosed in multiple embodiments may be used in a single embodiment. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. A surgical device for repairing tissue comprising:
a first anchor adapted to be implanted in a bone;
a second anchor adapted to be implanted in the bone; and
a continuous suture adapted to extend from the first anchor to the second anchor, wherein said continuous suture is adapted to wrap around tissue between the first anchor and the second anchor to secure the tissue between the first anchor and the second anchor.

2. The surgical device of claim 1, further comprising:
a third anchor adapted to be implanted in the bone, wherein the continuous suture is adapted to extend from the second anchor to wrap around the tissue between the second anchor and the third anchor.

3. The surgical device of claim 2, further comprising:
a fourth anchor adapted to be implanted in the bone, wherein the continuous suture is adapted to extend from the third anchor to wrap around the tissue between the third anchor and the fourth anchor.

4. The surgical device of claim 2, wherein the continuous suture is adapted to secure the tissue between the second anchor and the third anchor to the bone.

5. The surgical device of claim 3, wherein the continuous suture is adapted to secure the tissue between the third anchor and the fourth anchor to the bone.

6. The surgical device of claim 1, wherein the continuous suture partially wraps around the tissue.

7. The surgical device of claim 1, wherein the continuous suture is adapted to extend from a first side of the first anchor to a second side of the second anchor.

8. The surgical device of claim 2, wherein the continuous suture is adapted to extend from a second side of the second anchor to a first side of the third anchor.

9. The surgical device of claim 8, wherein the continuous suture comprises a separate suture that connects the second anchor to the third anchor.

10. A surgical apparatus for repairing tears in tissue comprising:
a first anchoring member adapted to be implanted in a bone;
a second anchoring member adapted to be implanted in the bone;
a continuous suture adapted to extend from the first anchor to the second anchor, wherein the continuous suture is adapted to wrap around the tissue between the first anchoring member and the second anchoring member to secure the tissue between the first anchoring member and the second anchoring member; and
a third anchoring member adapted to be implanted in the bone, wherein the continuous suture extends from the second anchoring member to wrap around tissue between the second anchoring member and the third anchoring member to secure the tissue between the second anchoring member and the third anchoring member.

11. The surgical apparatus of claim 10, further comprising:
a fourth anchoring member adapted to be implanted in the bone, wherein the continuous suture is adapted to extend from the third anchoring member to wrap around the tissue between the third anchoring member and the fourth anchoring member.

12. The surgical apparatus of claim 11, wherein the continuous suture is adapted to be attached to the fourth anchoring member to secure the tissue between the third anchoring member and the fourth anchoring member, where the continuous suture is an uninterrupted piece of suture that extends from the first anchoring member.

13. A surgical device for repairing tissue comprising:
a first anchor adapted to be implanted in a first location of a bone;
a second anchor adapted to be implanted in a second location of the bone fragment; and
a suture having a first end portion, a second end portion, and an intermediate portion, wherein the first end portion is configured to form a loop around the tissue at the first location, the second end portion configured to form a loop around the tissue at the second location, and the intermediate portion that extends from the first anchor to the second anchor wherein the intermediate portion is configured to wrap around the tissue between the first anchor and the second anchor to secure the tissue between the first anchor and the second anchor.

14. The surgical device of claim 13 wherein the suture is an uninterrupted suture that is continuous along its length from the first end portion to the second end portion.

15. The surgical device of claim 13 further comprising a second continuous suture that extends from the first anchor to the second anchor and that is configured to wrap around tissue.

16. The surgical device of claim 13 further wherein the first anchor comprises an aperture formed within a distal portion of the first anchor.

17. The surgical device of claim 16 wherein the first end portion of the continuous suture is disposed through the aperture of the distal portion of the first anchor.

18. The surgical device of claim 17 where second anchor comprises an aperture formed within a distal portion of the second anchor wherein the second end portion of the continuous suture is disposed through the aperture of the distal portion of the second anchor.

19. A method of repairing tissue, comprising:
passing a first suture around a tissue;
attaching a first end of the first suture to a first anchor;
implanting the first anchor at a first location of a bone;
wrapping a second end of the first suture around a tissue;
attaching the second end of the first suture to a second anchor; and
implanting the second anchor at a second location of the bone.

20. The method of claim 20, further comprising:
passing a second suture around a tissue;
attaching a first end of the second suture to the second anchor;
wrapping a second end of the second suture around the tissue;
attaching the second end of the second suture to a third anchor; and
implanting a third anchor at a third location of the bone.
